(19) United States
(12) Patent Application Publication
(10) Pub. No.: US 2006/0246595 A1
(43) Pub. Date: Nov. 2, 2006

(54) Method for using an all solid-state fluorometer in monitoring and controlling chemicals in water

(76) Inventors: Rodney H. Banks, Aurora, IL (US); Robert L. Wetegrove, Winfield, IL (US)

Correspondence Address: NALCO COMPANY, 1601 W. DIEHL ROAD, NAPERVILLE, IL, 60563-1198, US

(21) Appl. No.: 11/119,970

(22) Filed: May 2, 2005

(51) Int. Cl. G01N 33/00

(52) U.S. Cl. 436/86

(57) ABSTRACT

A method for monitoring and controlling the concentration of chemicals added to and present in water systems via the use of a solid state fluorometer. Biological materials that exist in water systems are monitored and controlled through the use of a solid state fluorometer.

### FIELD OF THE INVENTION

The present invention generally relates to the use of a solid-state fluorometer in a method for monitoring and controlling the concentration of chemicals added to and present in water systems.

### BACKGROUND OF THE INVENTION

U.S. Pat. No. 6,255,118 issued to Alfano et al. describes a method for monitoring and control of the concentration of chemicals in industrial systems by utilizing a solid-state fluorometer with an excitation source that is either a light emitting diode or a solid state diode laser and using said solid-state fluorometer to determine the concentration of a fluorescent tracer that is added to the industrial system in a known proportion to the chemical added to the industrial system. This patent is herein incorporated by reference.

### SUMMARY OF THE INVENTION

The present invention provides for a method for monitoring the concentration of one or more chemicals added to a water system. The method utilizes a solid state fluorometer that has one or more excitation sources that are either a light emitting diode or solid state laser diode. The light emitting diode emits light having a wavelength of from about 255 nm to about 365 nm or from about 520 nm to about 940 nm. The fluorometer also has one or more detectors that receive fluorescence from the excitation of the water system and produces an output signal proportional to the quantity of fluorescence received by the detectors. A fluorescent tracer is added to the water system in a known proportion to the chemical that is added to the water system. The chemical that is added to the water system may have fluorescent properties. The fluorometer as described above is used to detect the fluorescence of the fluorescent tracer or the fluorescence of the chemical that is added to the water system. The fluorometer is programmed to produce an output signal proportional to the detected fluorescence. Optionally, controlling dosage of the chemical added to the water system is based on the output signal from said fluorescent tracer or chemical detected by the fluorometer.

The present invention provides for a method for monitoring the concentration of one or more chemicals in a water system. The method utilizes a solid state fluorometer that has one or more excitation sources that are either a light emitting diode or solid state laser diode. The fluorometer has one or more detectors that receive fluorescence from the excitation of the water system and produce an output signal proportional to the quantity of fluorescence received by the detectors. The fluorometer is used to detect the fluorescence of the chemicals that are in the water system. The fluorometer is programmed to produce an output signal proportional to the detected fluorescence. Optionally, controlling the concentration of the chemicals in the water system is based on the output signal from said fluorescent chemical detected by the fluorometer.

The present invention is also directed to a method for fluorometric monitoring of one or more biological materials in a water system that utilize a solid state fluorometer that has one or more excitation sources that are either a light emitting diode or a solid state laser. The fluorometer has one or more detectors that receive fluorescence from the excitation of the water system and produce an output signal proportional to the quantity of fluorescence received by the detectors. The fluorometer as described above is used to detect the fluorescence of biological materials that are in the water system. The fluorometer is programmed to produce an output signal proportional to the detected fluorescence. Optionally, controlling the concentration of the biological materials in the water system is based on the output signal from said fluorescent biological materials detected by the fluorometer.

Additional features and advantages of the present invention are described in, and will be apparent from, the detailed description of the presently preferred embodiments.

### DESCRIPTION OF THE PREFERRED EMBODIMENTS

Throughout this patent application the following terms have the indicated meanings:

“About” means nearly or equal to.

“Water system” means a water system for consumer or industrial applications.

“NDSA” means naphthalene disulfonic acid.

“BSA” means bovine serum albumin.

“EWL” means egg white lysozyme.

“NADH” means nicotinamide adenine dinucleotide.

“NADPH” means nicotinamide adenine dinucleotide phosphate.

“LED” means light emitting diode.

“Biological materials” means living organisms or materials derived from living organisms.

“Chemical treatment” means a protocol invoking the addition of chemicals to a water system to produce a desired effect.

The solid-state diode laser or light emitting diode fluorometer instrument of the present invention is suitable for use in several industrial and consumer water applications. These include, but are not limited to, cooling water systems, boiler water systems, pulp slurries, ceramic slurries, waste-treatment, mining, agriculture, oil-field applications, drinking or potable water supplies, a reverse osmosis system, commercial and consumer hot water supplies and equipment, swimming pools and spas, amusement park rides, food processing and decorative fountains.

In one embodiment, the solid state fluorometer detector is a silicon photodiode. In another embodiment, the solid state fluorometer is a photomultiplier tube.

In another embodiment, the solid state fluorometer is used to monitor biological materials in a water system.

In yet another embodiment, the biological materials are selected from the group consisting of: amino acids; NADH; nucleic acids; tryptophan, tyrosine; adenine triphosphate; calcium dipicolinate; NAD(P)H; flavins; porphyrins; 3,4 dihydroxyphenyalanine; kyurenine; Serotonin; phenylalanine; dopamine; histamine; Vitamin A; p-aminobenzoic acid; Vitamin B12; estrogen; adenine diphosphate; adenine; adenosine; bovine serum albumin; egg white lysozyme; naphthalene disulfonic acid; microorganisms; toxins; spores; viruses; algae; fungi; and proteins.

For example, a 280 nm LED fluorometrically detects tryptophan, a common amino acid in proteins produced by living entities, the 340 nm LED detects NAD(P)H/NAD(P)⁺ ratios associated with cellular metabolism, and scattering can detect film deposit and biofilm formation.

In another embodiment, a fluorogenic dye is added to said water system. The fluorogenic dye reacts with a biological material in the water system. The reacted fluorogenic dye is analyzed with a fluorometer. U.S. Pat. Nos. 6,329,165 and 6,440,689 both describe this procedure and herein are incorporated by reference. In one embodiment, a 525 nm LED fluorometrically detects resazurin and resorufin.

In another embodiment, a chemical treatment is applied to a water system in response to the output signal from said chemical detected by said fluorometer. In one embodiment, the chemical treatment is a biocidal, biostatic, or other microorganism control agent. Both biocidal and biostatic control agents, include, but are not limited to the following compounds: hypohalous acids; halogen release compounds; halosulfamates; chlorine dioxide; ozone; peroxygen compounds; dibromonitrilopropionamide; isothiazolins; quaternary compounds, glutaraldehyde; triazines; and surfactants such as ethylene oxide/propylene oxide copolymers and polyalkylglycosides.

In another embodiment, the effective amount of chemical treatment is added to said water system to prevent microbial or contamination in said water system.

In another embodiment, biological materials fluoresce at an excitation wavelength from about 260 nm to about 350 nm.

Besides biological materials, other chemicals can be monitored such as fluorescent tracers used in water treatment. Some polymers and other chemical actives exhibit natural fluorescence in the region that solid-state LEDs or laser diodes emit. Devices of this invention can be made to measure DAXAD polymer or naphthalene sulfonic acid/formaldehyde sodium salt copolymer; NexGuard polymer or acrylic acid/styrene sulfonate copolymer and its lower molecular weight decomposition byproducts as well as naphthalene disulfonic acid, benzotriazole, tolyltriazole, hydroquinone, gallic acid, pyrogallol, sulfonated anthracenes, and fluorescently tagged polymer(s) which may be in the form of a concentration indicator (U.S. Pat. No. 5,435,969, which is herein incorporated by reference), or tagged polymers (U.S. Pat. Nos. 5,171,450 and 6,645,428 which are herein incorporated by reference).

The following examples are presented to describe preferred embodiment and utilization of the invention and are not meant to limit the invention unless otherwise stated in the claims appended hereto.

### EXAMPLES

### Example 1

Tryptophan is an amino acid that is one of the fluorescent components of proteins. Since proteins are essential elements of living things it is useful to be able to detect such fluorescent species if one is interested in detecting living organisms or residual protein contamination caused by living organisms. The following table shows the response of an LED-based detector to various low levels of the amino acid tryptophan.

TABLE ONE
Tryptophan Fluorescence with UV LED

| | Fluorescence with |
|---|---|
| Tryptophan μg/L | 280 nm Excitation |
| 0 | 22.6 |
| 10 | 34.65 |
| 100 | 151.3 |

### Example 2

One example of a protein containing fluorescent amino acids is bovine serum albumin (BSA), a component of cow's blood. The next table shows the proportional response to BSA from an LED-based fluorescence detector. This shows useful detection of protein that could be a component of biological fouling in an industrial system or for detecting protein contamination in meat processing equipment.

TABLE TWO
Bovine Serum Albumin Fluorescence with UV LED

| | Fluorescence with |
|---|---|
| Bovine Serum Albumin (mg/L) | 280 nm Excitation |
| 0 | 20.35 |
| 1 | 21 |
| 10 | 33.2 |
| 100 | 159.4 |

### Example 3

Another example of a protein containing fluorescent amino acids is egg white lysozyme, a component of hen's eggs. The next table shows detection of EWL with an LED-based fluorescence detector. This device could be used for detecting protein contamination in food processing or from microorganisms.

TABLE THREE
Egg White Lysozyme Fluorescence with UV LED

| | Fluorescence with |
|---|---|
| Egg White Lysozyme mg/L | 280 nm Excitation |
| 0 | 19.6 |
| 1 | 20.53 |
| 10 | 40.6 |
| 100 | 176.3 |

### Example 4

A component of living organisms is nicotinamide adenine dinucleotide. Known as NADH, this substance participates in chemical reduction and oxidation reactions in cells and is present in all living things, but is degraded rapidly after death. It is therefore useful to be able to detect NADH as an indicator of the presence of living organisms in biological contamination. The next chart shows detection of NADH by an LED detector with fluorescence excitation at 340 nm.

TABLE FOUR
NADH Fluorescence with UV LED

| | Fluorescence with |
|---|---|
| NADH μmoles/L | 340 nm Excitation |
| 0 | 0 |
| 5 | 30 |
| 25 | 157 |
| 50 | 262 |

### Example 5

The following table and chart show use of the LED fluorescence detection system to examine a series of samples containing levels of living Pseudomonas aeruginosa bacteria. These data show a threshold of fluorescence detection of about one thousand bacteria per mL at excitation wavelengths of 280 nm and 340 nm.

TABLE FIVE
Living bacteria count (CFU) and fluorescence
at 280 nm and 340 nm

| | Fluorescence with | Fluorescence with |
|---|---|---|
| log10 cfu/ml | 280 nm Excitation (mV) | 340 nm Excitation (mV) |
| 1.60E+01 | 21.3 | 8.5 |
| 1.60E+02 | 21.3 | 12.5 |
| 1.60E+03 | 24.2 | 60 |
| 1.61E+04 | 43.5 | 477 |

### Example 6

The following table shows data for fluorescence of NDSA using the 280 nm LED.

TABLE SIX
NDSA

| | Fluorescence with |
|---|---|
| NDSA (ppb) | 280 nm Excitation (mV) |
| 0 | 140 |
| 250 | 295 |
| 500 | 460 |
| 700 | 580 |

What is claimed is:

1. A method for monitoring the concentration of one or more chemicals added to a water system, the method comprising the steps of:
a) providing a solid state fluorometer, wherein said fluorometer comprises:
i) one or more solid-state excitation sources to direct light in a specified direction, wherein said excitation source is a light emitting diode or solid state laser diode, with said light emitting diode emitting light having a wavelength of from about 255 nm to about 365 nm or from about 520 nm to 940 nm, or with said solid state laser diode having a wavelength of from about 340 to about 600 nm;
ii) one or more detectors receiving the fluorescence from the excitation of the sample and producing an output signal proportional to the quantity of fluorescence received by the detector;
b) providing a water system, wherein said fluorescent tracer is added to said water system in a known proportion to said chemical or wherein said chemical that is added to said water system has fluorescent properties;
c) using said fluorometer to detect the fluorescence of the fluorescent tracer or fluorescence of the chemical treatment agent in said water system;
d) programming said fluorometer to produce an output signal proportional to the detected fluorescence; and

2. The method of claim 1 further comprising the step of controlling dosage of said chemical added to said water system based on said output signal from said fluorescent tracer or said chemical detected by said fluorometer.

3. The method of claim 1 wherein said chemicals are added to said water system selected from the group consisting of: naphthalene sulfonic acid/formaldehyde sodium salt copolymer; acrylate/styrene sulfonate copolymer and its decomposition by products lower molecular weight polymers or desulfonated polymers, as well as naphthalene disulfonic acid, benzotriazole, tolyltriazole, hydroquinone, gallic acid, pyrogallol, sulfonated anthracenes, and fluorescently tagged polymer.

4. The method of claim 1 wherein said water system are selected from the group consisting of a reverse osmosis system; a cooling water system, a boiler water system, pulp slurries, ceramic slurries, waste-treatment, mining, agriculture, oil-field applications, drinking or potable water supplies, a reverse osmosis system, commercial and consumer hot water supplies and equipment, swimming pools and spas, amusement park rides, food processing and decorative fountains.

5. A method for monitoring concentration of one or more chemicals in a water system, the method comprising the steps of:
a) providing a solid state fluorometer, wherein said fluorometer comprises:
i) one or more solid-state excitation sources to direct light in a specified direction, wherein said excitation source is a light emitting diode or a solid state laser diode.
ii) one or more detectors receiving the fluorescence from the excitation of the sample and producing an output signal proportional to the quantity of fluorescence received on the detector;
b) providing a water system;
c) using said fluorometer to detect the fluorescence of said chemicals in said water system;
d) programming said fluorometer to produce an output signal proportional to the detected fluorescence

6. The method of claim 5 wherein said light emitting diode or a solid state laser diode emits light having a wavelength from about 260 nm to about 350 nm.

7. The method of claim 5 further comprising the step of controlling concentration of said chemicals in said water system based on said output signal from said fluorescent chemicals detected by said fluorometer.

8. The method of claim 5 further comprising the step of providing an effective amount of chemical treatment to said water system in response to the output signal from said chemical detected by said fluorometer.

9. The method of claim 5 wherein said chemicals are biological materials.

10. The method of claim 9 wherein said biological materials are selected from the group consisting of: amino acids; NADH; nucleic acids; tryptophan, tyrosine; adenine triphosphate; calcium dipicolinate; NAD(P)H; flavins; porphyrins; 3,4 dihydroxyphenyalanine; kyurenine; Serotonin; phenylalanine; dopamine; histamine; Vitamin A; p-aminobenzoic acid; Vitamin B12; estrogen; adenine diphosphate; adenine; adenosine; bovine serum albumin; egg white lysozyme; naphthalene disulfonic acid; microorganisms; toxins; spores; viruses; algae; fungi; and proteins.

11. The method of claim 8 where said chemical treatment is selected from the group consisting of: a biocidal control agent, a biostatic control agent, and microorganism control agent.

12. The method of claim 1 wherein said water system are selected from the group consisting of a reverse osmosis system; a cooling water system, a boiler water system, pulp slurries, ceramic slurries, waste-treatment, mining, agriculture, oil-field applications, drinking or potable water supplies, a reverse osmosis system, commercial and consumer hot water supplies and equipment, swimming pools and spas, amusement park rides, food processing, and decorative fountains.

13. The method of claim 1 wherein said detector is a silicon photodiode.

14. The method of claim 5 wherein said detector is a silicon photodiode.

15. The method of claim 1 wherein said detector is a photomultiplier tube.

16. The method of claim 5 wherein said detector is a photomultiplier tube.

17. The method of claim 8 wherein said effective amount of chemical treatment is added to said water system to prevent microbial or contamination in said water system.

18. The method of claim 1 1 wherein said biocidal control agents and biostatic control agents are selected from the group consisting of: hypohalous acids; halogen release compounds; halosulfamates; chlorine dioxide; ozone; peroxygen compounds; dibromonitrilopropionamide; isothiazolins; quaternary compounds, glutaraldehyde; triazines; surfactants, ethylene oxide/propylene oxide copolymers; and polyalkylglycosides.

19. The method of claim 1 wherein said chemical added to said water system is a fluorogenic dye that can react with biological materials present in said water system.

20. The method of claim 19 wherein said fluorogenic dye is selected from the group consisting of: resazurin; and resorufin.
